(19) United States
(12) Patent Application Publication
(10) Pub. No.: US 2024/0200008 A1
(43) Pub. Date: Jun. 20, 2024

(54) DEVICE FOR MANUFACTURING CELL-DERIVED VESICLES AND MANUFACTURING METHOD USING SAME

(71) Applicant: MDimune Inc., Seoul (KR)

(72) Inventors: Jik Han JUNG, Seoul (KR); Byoung Jin KIM, Seoul (KR); Shin Gyu BAE, Seoul (KR)

(73) Assignee: MDimune Inc., Seoul (KR)

(21) Appl. No.: 18/593,346

(22) Filed: Mar. 1, 2024

Related U.S. Application Data
(63) Continuation of application No. PCT/KR2022/006155, filed on Apr. 29, 2022.
(60) Provisional application No. 63/240,215, filed on Sep. 2, 2021.

(51) Int. Cl. C12M 1/00; C12M 1/12; C12M 1/26

(52) U.S. Cl. CPC C12M 23/58; C12M 23/38; C12M 29/14; C12M 29/24; C12M 33/04; C12M 37/02

(57) ABSTRACT

The present invention relates to a method for manufacturing cell-derived vesicles using a depth filter. The present invention has the effect of enabling cell-derived vesicles to be manufactured stably, economically, and in large quantities through a simplified process.

### CROSS-REFERENCE TO RELATED APPLICATION

This application is a continuation of International Application No. PCT/KR2022/006155 filed Apr. 29, 2022 which claims priority to U.S. Provisional Application No. 63/240,215 filed Sep. 2, 2021, the entire disclosures of which are incorporated herein by reference.

### TECHNICAL FIELD

The present invention relates to a method for manufacturing cell-derived vesicles, and more particularly, to a method for manufacturing cell-derived vesicles for manufacturing cell-derived vesicles by transferring a fluid containing nucleated or non-nucleated cells into a depth filter using a pressure vessel, and a device for manufacturing cell-derived vesicles having a depth filter.

### BACKGROUND ART

Extracellular vesicles refer to various types of particles secreted by cells, and may be largely divided into exosomes derived from an endosomal pathway and microvesicles derived from a plasma membrane.

The exosomes, which are spherical vesicles released by cells, contain various information such as proteins and DNA of parent cells, and the development of cancer diagnostic markers and sensors by using the exosomes as biomarkers has been actively conducted.

In addition, the microvesicles are a type of cell organelles that is generally 0.03 to 1 μm in size and are naturally released from the cell membrane to have the form of a double phospholipid membrane, and contain cytoplasmic components such as mRNA, DNA, and proteins.

Recently, research on drug delivery systems using these extracellular vesicles has been actively conducted. As a trace amount of drug may be encapsulated in vesicles to deliver the corresponding drug to a specific site, the usability of extracellular vesicles is increasing in various medical fields. In particular, the microvesicles are directly released from the cell membrane and retain antigens of the parent cell as they are, so that the usability in vaccines, etc. is very high.

However, since the number of extracellular vesicles that may be obtained from cells is very limited, a method for manufacturing extracellular vesicle mimetics to artificially obtain a large amount has recently been required.

Typically, a centrifuge may be used as a method for obtaining extracellular vesicle mimetics, but the centrifuge is expensive and still has limitations in that the yield does not fall short of expectations.

### DISCLOSURE

### Technical Problem

An object of the present invention is to provide a method for manufacturing cell-derived vesicles using a depth filter capable of easily increasing a production scale and simplifying a production process in manufacturing the cell-derived vesicles.

### Technical Solution

An aspect of the present invention provides a method for manufacturing cell-derived vesicles using a depth filter including extruding a cell culture solution through a depth filter.

In addition, another aspect of the present invention provides a method for manufacturing cell-derived vesicles with reduced risk of exposure to external contamination, including a) harvesting cells by passing a cell culture solution containing the cells through a depth filter; b) resuspending the harvested cells by supplying a cell suspension; c) extruding the cells resuspended in step b) through a depth filter; and d) obtaining cell-derived vesicles from the extruded solution, in which steps a) to d) are performed in one device.

### Advantageous Effects

According to the present invention, there is the effect of enabling cell-derived vesicles to be manufactured stably, economically, and in large quantities through a simplified process.

### DESCRIPTION OF DRAWINGS

FIG. 1 is a diagram schematically illustrating a device for manufacturing cell-derived vesicles according to a first embodiment of the present invention.

FIG. 2 is a diagram schematically illustrating a device for manufacturing cell-derived vesicles according to a second embodiment of the present invention.

FIG. 3 is a diagram schematically illustrating a device for manufacturing cell-derived vesicles according to a third embodiment of the present invention.

FIG. 4 is a diagram schematically illustrating a device for manufacturing cell-derived vesicles according to a fourth embodiment of the present invention.

FIGS. 5 and 6 are exploded perspective view of a filter assembly.

FIG. 7 is a diagram illustrating trend results of production yield, particle concentration, particle size, PDI, protein concentration, and DNA concentration according to the number of extrusion times, when harvesting and extruding cells using the device for manufacturing the cell-derived vesicles according to the second embodiment of the present invention.

FIG. 8 is a diagram of results for the production yield of particles per cell according to various parameters.

FIG. 9 is a diagram of results for average time of once extruding process according to various parameters.

FIG. 10 is a diagram of results for production yield and average time of once extruding process according to a PBS amount per cell and a PBS temperature.

FIG. 11 is a diagram illustrating results of comparing shapes, size distributions, protein concentrations, DNA concentrations, PDIs, and average sizes between cell-derived vesicles manufactured by an extruding method using a membrane and cell-derived vesicles manufactured by an extruding method using a depth filter.

FIG. 12 is a diagram illustrating an extruder for manufacturing cell-derived vesicles including a depth filter (left: 5 L, right: 50 L).

### MODES OF THE INVENTION

Hereinafter, embodiments of the present invention will be described in detail with reference to the accompanying drawings. First, when reference numerals refer to components of each drawing, it is to be noted that although the same components are illustrated in different drawings, the same components are denoted by the same reference numerals as possible. In addition, if it is determined to obscure the gist of the present invention, the detailed description will be omitted. Further, hereinafter, a preferred embodiment of the present invention will be described, but the technical spirit of the present invention is not limited thereto or restricted thereby and the embodiments may be modified and variously executed by those skilled in the art.

The present invention relates to a method for manufacturing cell-derived vesicles using a depth filter, comprising a) extruding a cell culture solution through a depth filter; and b) obtaining cell-derived vesicles from the extruded solution.

The manufacturing method of the present invention further includes a-1) harvesting the cells by passing the cell culture solution containing the cells through the depth filter; and a-2) resuspending the harvested cells by supplying a cell suspension, before the extruding step of step a), in which the cells in step a) may be cells in the cell suspension.

The manufacturing method of the present invention may be applied to both suspension cells and adherent cells.

At this time, the harvesting step, the resuspending step, and the extruding step are performed in one device. When all manufacturing processes are performed in one device without filter exchange, the risk of exposure to external contamination may be minimized.

The extruding step may be performed by changing an extrusion pressure and may be performed by increasing the pressure of the second and subsequent extruding steps compared to the pressure of the first extruding step.

In the present invention, the extruding step of step a) may be performed at a higher pressure than the harvesting step of step a-1), and preferably, the harvesting step of step a-1) may be performed under pressure conditions of is 0.1 to 0.4 bar, and the extruding step of step a) may be performed under pressure conditions of 1 to 2.5 bar. That is, the pressure used for the extrusion may be using a higher pressure than the pressure of less than 1 bar commonly used when using a conventional depth filter for cell filtration. As such, the cell-derived vesicles with new characteristics may be manufactured from cells by applying the high pressure to the depth filter to perform an extrusion process rather than filtration.

The cell suspension of the present invention may be supplied in a reverse direction of the direction in which the cell culture solution is discharged from the depth filter.

In the present invention, the extruding step of step a) may be repeated 1 to 120 times, preferably 1 to 100 times, 1 to 80 times, and 1 to 7 times.

At this time, the repetition may be performed by reciprocating through one depth filter, and may be performed while changing the pressure.

The manufacturing method of the present invention may be performed by changing the pressure to 1 bar as a first pressure and 2.5 bar as a second pressure.

That is, according to the manufacturing process using the depth filter of the present invention, since the suspension may be extruded repeatedly based on one depth filter, there is an advantage that there is no need to change a filter, and there is no filter clogging, which is a disadvantage of the sequential extrusion process using a membrane filter.

A device having the depth filter used in the present invention may include a first pressure vessel and a second pressure vessel, on one side to which a culture solution supply line to which a cell culture solution is supplied and a gas supply line are connected; a first extrusion line connected to the other side of the first pressure vessel; a second extrusion line which is connected to the other side of the second pressure vessel and communicates with the first extrusion line; at least one depth filter disposed on the first extrusion line or the second extrusion line and through which the cell culture solution passes; and a harvesting line which communicates with the first extrusion line and the second extrusion line.

A device 10 for manufacturing cell-derived vesicles according to the first embodiment of the present invention includes a pressure vessel 100 and a filter 110.

Specifically, a culture solution supply line 102 through which the cell culture solution is supplied, a gas supply line 104, and a vent line 106 are connected to one side of the pressure vessel 100, and an extrusion line 112 is connected to the other side.

At least one filter 110 through which the cell culture solution discharged from the pressure vessel 100 passes is disposed on the path of the extrusion line 112, and a harvesting line 114 communicating with the extrusion line 112 is provided at the end of the extrusion line 112.

At this time, the filter 110 is preferably a depth filter.

At least one depth filter may be disposed on the first extrusion line and the second extrusion line.

Further, the present invention relates to a method for manufacturing cell-derived vesicles with reduced risk of exposure to external contamination, comprising a) harvesting cells by passing a cell culture solution containing the cells through a depth filter; b) resuspending the harvested cells by supplying a cell suspension; c) extruding the cells resuspended in step b) through the depth filter; and d) obtaining cell-derived vesicles from the extruded solution, in which steps a) to d) are performed in one device.

The manufacturing method may be performed in one device to reduce the risk of cells or cell-derived vesicles to be exposed to an external environment to be contaminated or deformed.

In addition, the manufacturing method is performed in one device to improve a process rate.

According to the manufacturing method of the present invention, the cell-derived vesicles are manufactured.

The cell-derived vesicles are manufactured through extrusion and thus distinguished from naturally secreted extracellular vesicles (EVs). Specifically, the cell-derived vesicles may include an extracellular organelle membrane in addition to the plasma membrane, and have an expression marker pattern that is different from that of EVs secreted by cells. Preferably, CDV manufactured through the extrusion process of the present invention is rich in LAMP1, Calnexin, and GM130, which are membrane proteins as a characteristic of intracellular organelles such as ERs, golgi bodies, and lysosomes, and may have an expression pattern of a tetraspanin marker such as CD9, CD63, and CD81, which is different from that of cell-secreted EV.

In addition, the cell-derived vesicles manufactured by the manufacturing method of the present invention may have a superior cell uptake ability to cell-secreted EVs obtained by a filtration separation process.

In addition, according to the manufacturing method of the present invention, it is possible to manufacture cell-derived vesicles having uniform shape, size distribution, protein and DNA concentrations, PDI, and average size of the cell-derived vesicles, which are industrially highly useful.

Hereinafter, a device of the present invention will be described in more detail.

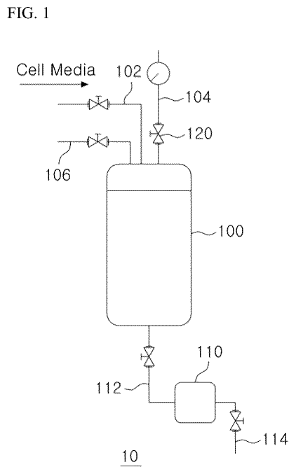

FIG. 1 is a diagram schematically illustrating a device for manufacturing cell-derived vesicles according to a first embodiment of the present invention.

Valves 120 are provided in the culture solution supply line 102, the gas supply line 104, the vent line 106, the extrusion line 112, and the harvesting line 114 included in the device of the present invention, respectively to control the opening and closing of the corresponding lines or the pressure of a fluid to be supplied or discharged.

Gas such as air or nitrogen is supplied from a separate gas tank (not shown) connected to the gas supply line 104 through the gas supply line 104.

A cell harvesting method for separating spent media from the cell culture solution using the device 10 for manufacturing the cell-derived vesicles according to the first embodiment of the present invention is as follows.

First, the cell culture solution is supplied into the pressure vessel 100 through the culture solution supply line 102. Thereafter, the gas supply line 104 connected to the gas tank is opened, and the vent line 106 is closed to allow the cell culture solution to pass through the filter 110 through the extrusion line 112 under a predetermined pressure, and the spent media without remaining cells are discharged through the harvesting line 114.

A method for manufacturing cell-derived vesicles using the device 10 for manufacturing the cell-derived vesicles according to the first embodiment of the present invention is as follows.

In the cell harvesting method, the following step is further performed after discharging the spent media.

First, while the gas supply line 104 is closed, a solution suitable for suspending cells, such as phosphate buffer saline (PBS), is injected through the harvesting line 114 to form a cell suspension in the pressure vessel 100. Thereafter, the gas supply line 104 connected to the gas tank is opened, and the vent line 106 is closed to allow the cell suspension in the pressure vessel 100 to pass through the filter 110 through the extrusion line 112 under a predetermined pressure, and a final extrusion solution is obtained through the harvesting line 114.

At this time, the final extrusion solution may contain cell-derived vesicles.

Meanwhile, in the method for manufacturing the cell-derived vesicles, the cell suspension instead of the cell culture medium may be supplied into the pressure vessel 100 through the culture solution supply line 102. At this time, the step of forming the cell suspension in the pressure vessel 100 by injecting the solution suitable for suspending the cells through the cell harvesting method and the harvesting line 114 may be omitted. Also, at this time, the cell suspension may be a solution obtained by harvesting cells from the cell culture solution through centrifugation and resuspending the cells in a solution suitable for suspending cells, such as PBS.

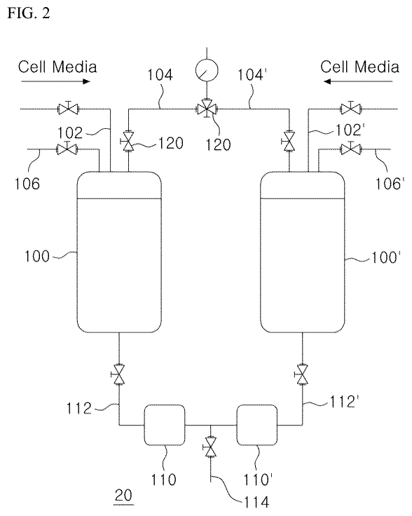

FIG. 2 is a diagram schematically illustrating a device for manufacturing cell-derived vesicles according to a second embodiment of the present invention.

A device 20 for manufacturing cell-derived vesicles according to the second embodiment of the present invention has a structure in which the device 10 for manufacturing the cell-derived vesicles according to the first embodiment described above is symmetrically connected.

Specifically, the device 20 for manufacturing cell-derived vesicles according to the second embodiment of the present invention includes a first pressure vessel 100 and a second pressure vessel 100′ of which culture solution supply lines 102 and 102′ to which a cell culture solution is supplied, gas supply lines 104 and 104′ and vent lines 106 and 106′ are connected to one side; a first extrusion line 112 connected to the other side of the first pressure vessel 100; a second extrusion line 112′ which is connected to the other side of the second pressure vessel 100′ and communicates with the first extrusion line 112; filters 110 and 110′ which are disposed on the first extrusion line 112 and the second extrusion line 112′ and through which the cell culture solution passes; and a harvesting line 114 which communicates with the first extrusion line 110 and the second extrusion line 112′.

In addition, valves 120 are provided in the culture solution supply lines 102 and 102′, the gas supply lines 104 and 104′, the vent lines 106 and 106′, the extrusion lines 112 and 112′, and the harvesting line 114, respectively to control the opening and closing of the corresponding lines or control the pressure of the supplied or discharged fluid.

At this time, the gas supply lines 104 and 104′ may communicate with each other to be connected to one gas tank (not shown), and adjust the flow direction and pressure of the supplied gas by adjusting the opening and closing and the degree of opening and closing of the valves 120.

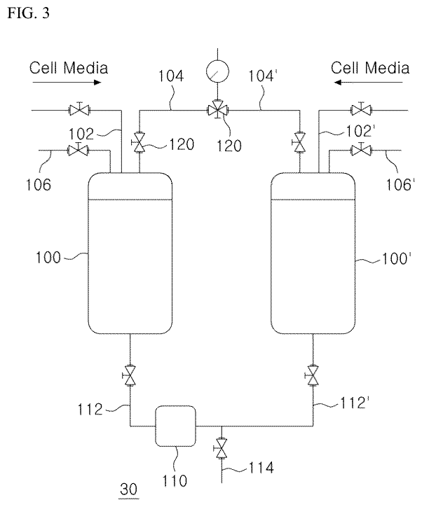

FIG. 3 is a diagram schematically illustrating a device for manufacturing cell-derived vesicles according to a third embodiment of the present invention.

A device 30 for manufacturing the cell-derived vesicles according to the third embodiment of the present invention has other components configured in the same manner as the device 20 for manufacturing the cell-derived vesicles according to the second embodiment of the present invention, except for providing only one filter 110 compared to the device 20 for manufacturing the cell-derived vesicles according to the second embodiment of the present invention.

A method for manufacturing the cell-derived vesicles using the device 20 or 30 for manufacturing the cell-derived vesicles according to the second or third embodiment of the present invention is similar to that of the first embodiment, but has a difference of using the first pressure vessel 100 and the second pressure vessel 100′. First, after the cell culture solution is added into at least one pressure vessel of the first pressure vessel 100 and the second pressure vessel 100′ through the culture solution supply lines 102 and 102′, a cell suspension in the pressure vessels 100 and 100′ is formed by performing the extrusion and harvesting in the same manner as the first embodiment.

Next, the gas supply line 104 connected to the first pressure vessel 100 is opened and the vent line 106 is closed. In addition, the gas supply line 104′ provided in the second pressure vessel 100′ is closed and the vent line 106′ is opened so that the cell suspension in the first pressure vessel 100 passes through the filters 110 and 110′ via the first extrusion line 112 and then moves to the second pressure vessel 100′.

Thereafter, contrary to the previous step, the gas supply line 104 connected to the first pressure vessel 100 is closed and the vent line 106 is opened. In addition, the gas supply line 104′ provided in the second pressure vessel 100′ is opened and the vent line 106′ is closed, so that the cell suspension which has been contained in the second pressure vessel 100′ flows in an opposite direction to the previous step, that is, toward the first pressure vessel 100 from the second pressure vessel 100′ to pass through the filters 110 and 110′.

The process of alternately opening and closing the gas supply lines 104 and 104′ as described above is repeated the predetermined number of times to allow the cell suspension to reciprocate through the filters 110 and 110′ multiple times, thereby increasing the production efficiency of the cell-derived vesicles.

After the cell suspension passes through the filters 110 and 110′ repeatedly the predetermined number of times, the harvesting line 114 is opened to obtain the final extrusion solution.

Meanwhile, in the manufacturing method, when manufacturing the cell-derived vesicles using the device 30 for manufacturing the cell-derived vesicles according to the third embodiment of the present invention, in the step of introducing the cell culture solution into the pressure vessel, the cell culture solution may be introduced into the pressure vessel adjacent to the filter 110 of the first pressure vessel 100 and the second pressure vessel 100′.

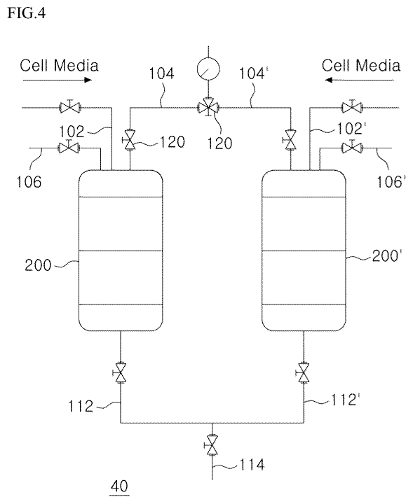

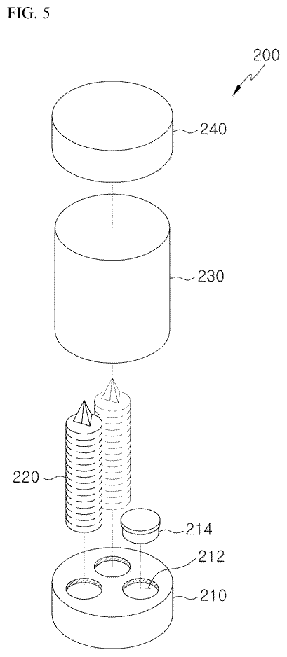

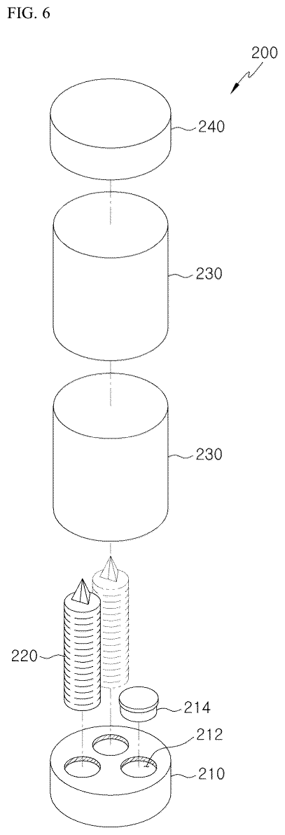

FIG. 4 is a diagram schematically illustrating a device for manufacturing cell-derived vesicles according to a fourth embodiment of the present invention, and FIGS. 5 and 6 are exploded perspective views of a filter assembly.

A device 40 for manufacturing cell-derived vesicles according to a fourth embodiment of the present invention includes a first filter assembly 200 and a second filter assembly 200′ of which culture solution supply lines 102 and 102′ to which a cell culture solution is supplied, gas supply lines 104 and 104′, and vent lines 106 and 106′ are connected to one side and in which a filter 220 is provided; a first extrusion line 112 connected to the other side of the first filter assembly 200; a second extrusion line 112′ which is connected to the other side of the second filter assembly 200′ and communicates with the first extrusion line 112; and a harvesting line 114 which communicates with the first extrusion line 112 and the second extrusion line 112′.

In addition, valves 120 are provided in the lines 102, 102′, 104, 104′, 106, 106′, 112, 112′, and 114, respectively, and the connection relationship thereof is configured in the same manner as that of the devices 20 and 30 for manufacturing the cell-derived vesicles according to the second and third embodiments.

However, in the device 40 for manufacturing the cell-derived vesicles according to the fourth embodiment, a filter 220 is not provided separately on the extrusion lines 112 and 112′, but is provided within the filter assemblies 200 and 200′, which is different from the devices 20 and 30 of the second and third embodiments.

Specifically, the filter assemblies 200 and 200′ include a lower portion 210 in which at least one mounting hole 212 is formed, a cartridge filter 220 detachably provided in the mounting hole 212, a middle portion 230 which is formed so that the cartridge filter 220 is contained therein and coupled to the lower portion 210, and an upper portion 240 which is fastened to the upper end of the middle portion 230 and seals the inside together with the lower portion 210 and the middle portion 230.

Here, the lower portion 210, the middle portion 230, and the upper portion 240 are coupled to each other and serve as a pressure vessel with a sealed interior, and the cell culture solution supplied to the inside is filtered or extruded through the cartridge filter 220.

At this time, the extrusion lines 112 and 112′ are connected to the lower portion 210, and the culture solution supply lines 102 and 102′, the gas supply lines 104 and 104′, and the vent lines 106 and 106′ are connected to the upper portion 240.

In addition, as illustrated in FIG. 6, the filter assemblies 200 and 200′ may include a plurality of middle portions 230 that are coupled to or separated from each other. The plurality of middle portions 230 are coupled to each other to expand an internal space of the filter assemblies 200 and 200′, and in this way, the extrusion scale and manufacturing capacity may be easily adjusted as needed.

In general, the maximum acceptable capacity of the pressure vessels 100 and 100′ is 2 L and the applicable filter area is 0.026 m² to 0.1 m², while the acceptable capacity of the filter assemblies 200 and 200′ is 5 L to 30 L and the applicable filter area is 0.45 m² to 2.7 m². There is an advantage of increasing the production yield in that the maximum capacity of the filter assemblies 200 and 200′ may be further increased according to an increase in the quantity of the coupled middle portions 230 or the expansion of the lower portion 210, and the applicable filter area may be further increased according to an increase in the mounting holes 212.

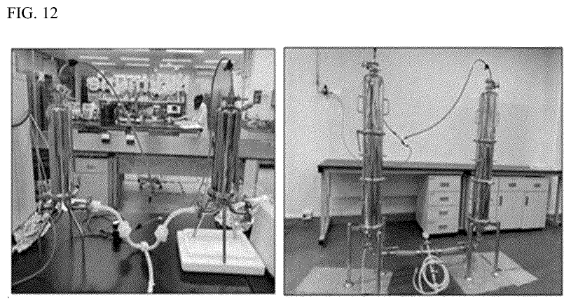

In the manufacturing method of the present invention, cell-derived vesicles may be manufactured by extruding a cell culture solution preferably with a capacity of 50 L to several thousands L. An extruder having the depth filter is shown in FIG. 12.

Meanwhile, the cartridge filters 220 may be mounted in the mounting holes 212 formed in the lower portion 210 as many as the number of mounting holes 212 formed, but if necessary, fewer cartridge filters 220 than the number of mounting holes 212 may be mounted.

In this case, in order to ensure smooth extrusion performance of the cell culture solution, it is preferred to close an idle mounting hole 212 where the cartridge filter 220 is not mounted. To this end, the filter assemblies 200 and 200′ may further include a cap 214 for closing the idle mounting hole 212.

The cap 214 is provided to be detachable from the mounting hole 212 and is formed to seal the mounting hole 212 when inserted into the mounting hole 212.

In addition, the filter assemblies 200 and 200′ may further include a pressure regulator which is connected to the gas supply lines 104 and 104′ and provided for gas mixing and pressure control.

Although not particularly shown, the device 40 for manufacturing the cell-derived vesicles according to the fourth embodiment of the present invention may be formed in a structure in which three or more filter assemblies 200 are connected to each other in parallel.

In this case, the added filter assembly 200 is provided with a culture solution supply line 102 and a vent line 106, a gas supply line 104 connected to the gas tank and the harvesting line 114, respectively, and an extrusion line 112, as described above.

Hereinafter, Experimental Examples performed using the devices 10, 20, 30, and 40 for manufacturing the cell-derived vesicles according to the first to fourth embodiments of the present invention will be described.

In Experimental Examples, particle concentration and size, PDI, and protein and DNA concentrations were commonly measured as follows.

### Measurement of Particle Concentration and Size

The concentration and size of particles were measured using Zetaview equipment (Particle matrix). Equipment preparation and cleaning processes were performed according to the manufacturer's instructions. A sample prepared for analysis was diluted using a PBS solution passing through a filter with pores of 0.1 μm to be 50 to 200 particles/frame, and particle concentration and particle size were measured using 1 mL of the sample.

### Measurement of PDI

PDI was measured using Zatasizer NanoZS (Malvern). The prepared sample was diluted using a PBS solution passing through a filter with pores of 0.1 μm to be a derived count rate of 600 to 1200, and then 1 to 2 mL of the sample was placed in a cuvette (Kartell) and the PDI was measured.

### Measurement of Protein Concentration and DNA Concentration

Protein and DNA concentrations were measured using a Qubit™ 4 Fluorometer (Invitrogen). For the prepared sample, the protein and DNA concentrations were measured using a Qubit™ Protein Assay Kit and a Qubit™ dsDNA HS Assay Kit, respectively. The sample and a standard solution were mixed with a working solution by vortexing, and a solution for protein measurement was reacted at room temperature for 15 minutes and a solution for DNA measurement was reacted for 2 minutes. Thereafter, each reacted solution was placed in a Qubit™ 4 Fluorometer chamber and the final concentration was measured according to the manufacturer's instructions.

A test for harvesting cells was conducted as in Experimental Example 1 below using the device 10 for manufacturing the cell-derived vesicles according to the first embodiment of the present invention.

### Experimental Example 1

For cell harvesting, one depth filter (Sartopure PP3 Capsules, total membrane area: 0.026 m², retention rates: 0.65 μm) was connected to a pressure vessel, and a gas supply line for supplying air was connected to the pressure vessel. 500 mL of a HEK293 cell culture solution at a concentration of 5.78E+06 cells/mL was added to the pressure vessel. The spent media were discharged to a harvest line by pressurizing with 0.1 bar of air. The discharge time of the spent media was 120 seconds.

To analyze whether cells remained in the spent media, 5 to 10 mL was sampled after sufficiently mixing the spent media physically, and the sample was evenly distributed on the surface of a 100 mm culture petri-dish for cell observation. Then, the sample was visually inspected using an Olympus CKX53 inverted microscope at 4×, 10×, and 40× magnifications. As a result, it was analyzed that there were no cells remaining in the spent media.

A cell extruding process was performed as in Experimental Examples 2 to 17 below using the device 20 for manufacturing the cell-derived vesicles according to the second embodiment of the present invention.

### Experimental Example 2

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 500 mL of a HEK293 cell culture solution at a concentration of 7.87E+06 cells/mL was centrifuged and harvested. The harvested cells were resuspended in 500 mL of room-temperature phosphate buffer saline (PBS) and then added to the two pressure vessels in half. The resuspended cell solution passed through the filter in one direction by closing one valve of the gas supply line and opening the other valve thereof. The resuspended cell solution passed through the filter total 60 times by repeating the opening and closing of the left and right valves in reverse. The air pressure supplied to the pressure vessel was 1 bar for the first extrusion and 2.5 bar for extrusion 2 to 60 times. The particle concentration in the final extrusion solution was measured, and the production yield of particles per cell was analyzed as 11,563 particles/cell.

### Experimental Example 3

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 500 mL of a HEK293 cell culture solution at a concentration of 7.65E+06 cells/mL was centrifuged and harvested. The harvested cells were resuspended in 500 mL of room-temperature PBS and then added to the two pressure vessels in half. The resuspended cell solution passed through the filter in one direction by closing one valve of the gas supply line and opening the other valve thereof. The resuspended cell solution passed through the filter total 100 times by repeating the opening and closing of the left and right valves in reverse. The air pressure supplied to the pressure vessel was 1 bar for the first extrusion and 2.5 bar for extrusion 2 to 100 times. The particle concentration in the final extrusion solution was measured, and the production yield of particles per cell was analyzed as 17,300 particles/cell.

### Experimental Example 4

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 300 mL of a HEK293 cell culture solution at a concentration of 5.80E+06 cells/mL was centrifuged and harvested. The harvested cells were resuspended in 500 mL of room-temperature PBS and then added to the two pressure vessels in half. The resuspended cell solution passed through the filter in one direction by closing one valve of the gas supply line and opening the other valve thereof. The resuspended cell solution passed through the filter total 90 times by repeating the opening and closing of the left and right valves in reverse. The air pressure supplied to the pressure vessel was 1 bar for the first extrusion and 2.5 bar for extrusion 2 to 90 times. The particle concentration in the final extrusion solution was measured, and the production yield of particles per cell was analyzed as 17,241 particles/cell.

### Experimental Example 5

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 584 mL of a HEK293 cell culture solution at a concentration of 3.15E+06 cells/mL was added to the two pressure vessels in half. The spent media were discharged to a harvest line by opening both valves of the gas supply line and pressurizing at 0.2 bar. The discharge time of the spent media was 29 seconds. To analyze whether cells remained in the spent media, 5 to 10 mL was sampled after sufficiently mixing the spent media physically, and the sample was evenly distributed on the surface of a 100 mm culture petri-dish for cell observation. Next, the sample was visually inspected using an Olympus CKX53 inverted microscope at 4×, 10×, and 40× magnifications, and it was analyzed that there were no cells remaining in the spent media. Back-flushing, a process of resuspending the harvested cells inside an extrusion device, was performed by back-injecting 300 mL of room-temperature PBS into a harvest line using a peristaltic pump. The back-flushing time was total 81 seconds. For extrusion, the resuspended cell solution passed through the filter in one direction by closing one valve of the gas supply line and opening the other valve thereof. The resuspended cell solution passed through the filter total 120 times by repeating the opening and closing of the left and right valves in reverse. The air pressure supplied to the pressure vessel was set to 1.0 bar. The particle concentration in the final extrusion solution was measured, and the production yield of particles per cell was analyzed as 15,492 particles/cell.

### Experimental Example 6

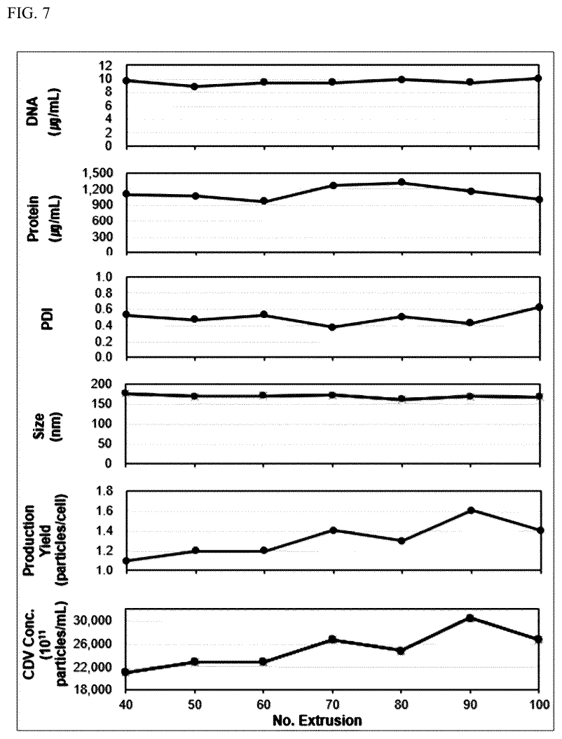

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 500 mL of a HEK293 cell culture solution at a concentration of 3.15E+06 cells/mL was added to the two pressure vessels in half. The spent media were discharged to a harvest line by opening both valves of the gas supply line and pressurizing at 0.2 bar. The discharge time of the spent media was 30 seconds. As a result of analyzing the spent media using the method for analyzing whether cells remained in the spent media described in Experimental Example 5, it was analyzed that there were no cells remaining in the spent media. 300 mL of room-temperature PBS was back-flushed into the harvest line using a peristaltic pump. The back-flushing time was total 90 seconds. For extrusion, the resuspended cell solution passed through the filter in one direction by closing one valve of the gas supply line and opening the other valve thereof. The resuspended cell solution passed through the filter total 100 times by repeating the opening and closing of the left and right valves in reverse. The air pressure supplied to the pressure vessel was 1.0 bar for the first extrusion and 2.5 bar for extrusion 2 to 100 times. The particle concentration, particle size, PDI, protein concentration, and DNA concentration in the final extrusion solution were measured, and the production yield of particles per cell was analyzed as 30,476 particles/cell. The analysis results thereof were as shown in FIG. 7.

### Experimental Example 7

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 400 mL of a HEK293 cell culture solution at a concentration of 7.62E+06 cells/mL was added to the two pressure vessels in half. The spent media were discharged to a harvest line by opening both valves of the gas supply line and pressurizing at 0.2 bar. The discharge time of the spent media was 33 seconds. As a result of analyzing the spent media using the method for analyzing whether cells remained in the spent media described in Experimental Example 5, it was analyzed that there were no cells remaining in the spent media. 400 mL of room-temperature PBS was back-flushed into the harvest line using a peristaltic pump. The back-flushing time was total 120 seconds. For extrusion, the resuspended cell solution passed through the filter in one direction by closing one valve of the gas supply line and opening the other valve thereof. The resuspended cell solution passed through the filter total 80 times by repeating the opening and closing of the left and right valves in reverse. The air pressure supplied to the pressure vessel was 1.0 bar for the first extrusion and 2.5 bar for extrusion 2 to 80 times. The particle concentration in the final extrusion solution was measured, and the production yield of particles per cell was analyzed as 23,622 particles/cell.

### Experimental Example 8

For cell extrusion, two depth filters (Sartopure PP3 Capsules, total membrane area: 0.052 m², retention rates: 0.65 μm) were connected between pressure vessels, and a gas supply line for supplying air was connected to each pressure vessel. 390 mL of a HEK293 cell culture solution at a concentration of 7.52E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 200 mL of room-temperature PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 80 times by repeating the opening and closing of the left and right valves in reverse. The air pressure supplied to the pressure vessel was 1.0 bar for the first extrusion and 2.5 bar for extrusion 2 to 80 times. The particle concentration in the final extrusion solution was measured, and the production yield of particles per cell was analyzed as 12,275 particles/cell.

### Experimental Example 9

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 867 mL of a HEK293 cell culture solution at a concentration of 3.46E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 400 mL of room-temperature PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 36 seconds. The particle concentration in the final extrusion solution was measured, and the production yield of particles per cell was analyzed as 24,001 particles/cell.

### Experimental Example 10

For cell extrusion, two depth filters (Sartopure PP3 MiDicaps, total membrane area: 0.1 m², retention rates: 0.65 μm) were connected between pressure vessels, and a gas supply line for supplying nitrogen (N₂) gas was connected to each pressure vessel. 643 mL of a HEK293 cell culture solution at a concentration of 8.13E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 800 mL of room-temperature PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 50 times by repeating the opening and closing of the left and right valves in reverse. The nitrogen gas pressure supplied to the pressure vessel was 1.0 bar for the first extrusion and 2.5 bar for extrusion 2 to 50 times. The average time required for one extrusion was measured as 56 seconds. The particle concentration in the final extrusion solution was measured, and the production yield of particles per cell was analyzed as 22,955 particles/cell.

### Experimental Example 11

For cell extrusion, two depth filters (Sartopure PP3 MiDicaps, total membrane area: 0.1 m², retention rates: 0.65 μm) were connected between pressure vessels, and a gas supply line for supplying nitrogen (N₂) gas was connected to each pressure vessel. 793 mL of a HEK293 cell culture solution at a concentration of 8.09E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 800 mL of room-temperature PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 47 seconds. The particle concentration in the final extrusion solution was measured, and the production yield of particles per cell was analyzed as 34,916 particles/cell.

### Experimental Example 12

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 575 mL of a HEK293 cell culture solution at a concentration of 5.22E+06 cells/mL was added to the two pressure vessels in half. The spent media were discharged to a harvest line by opening both valves of the gas supply line and pressurizing at 0.2 bar. The discharge time of the spent media was 4 minutes. As a result of analyzing the spent media using the method for analyzing whether cells remained in the spent media described in Experimental Example 5, it was analyzed that there were no cells remaining in the spent media. 800 mL of room-temperature PBS was back-flushed into the harvest line using a peristaltic pump. The back-flushing time was total 2 minutes. For extrusion, the resuspended cell solution passed through the filter in one direction by closing one valve of the gas supply line and opening the other valve thereof. The resuspended cell solution passed through the filter total 80 times by repeating the opening and closing of the left and right valves in reverse. The nitrogen gas pressure supplied to the pressure vessel was 1.0 bar for the first extrusion and 2.5 bar for extrusion 2 to 80 times. The particle concentration in the final extrusion solution was measured, and the production yield of particles per cell was analyzed as 18,124 particles/cell.

### Experimental Example 13

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 575 mL of a HEK293 cell culture solution at a concentration of 5.22E+06 cells/mL was added to the two pressure vessels in half. The spent media were discharged to a harvest line by opening both valves of the gas supply line and pressurizing at 0.2 bar. The discharge time of the spent media was 4 minutes. As a result of analyzing the spent media using the method for analyzing whether cells remained in the spent media described in Experimental Example 5, it was analyzed that there were no cells remaining in the spent media. 1,600 mL of room-temperature PBS was back-flushed into the harvest line using a peristaltic pump. The back-flushing time was total 4 minutes. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The particle concentration in the final extrusion solution was measured, and the production yield of particles per cell was analyzed as 23,389 particles/cell.

### Experimental Example 14

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 575 mL of a HEK293 cell culture solution at a concentration of 5.22E+06 cells/mL was added to the two pressure vessels in half. The spent media were discharged to a harvest line by opening both valves of the gas supply line and pressurizing at 0.2 bar. The discharge time of the spent media was 4 minutes. As a result of analyzing the spent media using the method for analyzing whether cells remained in the spent media described in Experimental Example 5, it was analyzed that there were no cells remaining in the spent media. 800 mL of 37° C. PBS was back-flushed into the harvest line using a peristaltic pump. The back-flushing time was total 2 minutes. For extrusion, the resuspended cell solution passed through the filter in one direction by closing one valve of the gas supply line and opening the other valve thereof. The resuspended cell solution passed through the filter total 80 times by repeating the opening and closing of the left and right valves in reverse. The nitrogen gas pressure supplied to the pressure vessel was 1.0 bar for the first extrusion and 2.5 bar for extrusion 2 to 80 times. The particle concentration in the final extrusion solution was measured, and the production yield of particles per cell was analyzed as 23,988 particles/cell.

### Experimental Example 15

For cell extrusion, two depth filters (Sartopure® PP3 Capsules, total membrane area: 0.052 m², retention rates: 0.65 μm) were connected between pressure vessels, and a gas supply line for supplying nitrogen (N₂) gas was connected to each pressure vessel. 658 mL of a HEK293 cell culture solution at a concentration of 4.56E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 1,600 mL of 37° C. PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 68 seconds. The particle concentration in the final extrusion solution was measured, and the production yield of particles per cell was analyzed as 26,146 particles/cell.

### Experimental Example 16

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 684 mL of a HEK293 cell culture solution at a concentration of 4.38E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 800 mL of 37° C. PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 37 seconds. The particle concentration in the final extrusion solution was measured, and the production yield of particles per cell was analyzed as 23,800 particles/cell.

### Experimental Example 17

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. 667 mL of a HEK293 cell culture solution at a concentration of 4.80E+06 cells/mL was added to the two pressure vessels in half. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 400 mL of 37° PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 21 seconds. The particle concentration in the final extrusion solution was measured, and the production yield of particles per cell was analyzed as 16,374 particles/cell.

A cell extruding process was performed as in Experimental Example 18 below using the device 30 for manufacturing the cell-derived vesicles according to the third embodiment of the present invention.

### Experimental Example 18

For cell extrusion, one depth filter (Sartopure PP3 MiDicaps, total membrane area: 0.05 m², retention rates: 0.65 μm) was connected between pressure vessels, and a gas supply line for supplying nitrogen (N₂) gas was connected to each pressure vessel. 620 mL of a HEK293 cell culture solution at a concentration of 3.72E+06 cells/mL was added to the pressure vessel connected to the depth filter in a forward direction. The spent media were discharged to a harvest line by opening both valves of the gas supply line and pressurizing at 0.2 bar. As a result of analyzing the spent media using the method for analyzing whether cells remained in the spent media described in Experimental Example 5, it was analyzed that there were no cells remaining in the spent media. 615 mL of 37° ° C. PBS was back-flushed into the harvest line using a peristaltic pump. For extrusion, the resuspended cell solution passed through the filter in one direction by closing one valve of the gas supply line and opening the other valve thereof. The resuspended cell solution passed through the filter total 70 times by repeating the opening and closing of the left and right valves in reverse. The nitrogen gas pressure supplied to the pressure vessel was 1.0 bar for the first extrusion and 2.5 bar for extrusion 2 to 70 times. The average time required for one extrusion was measured as 37 seconds. The particle concentration in the final extrusion solution was measured, and the production yield of particles per cell was analyzed as 29,343 particles/cell.

A cell extruding process was performed as in Experimental Examples 19 to 22 below using the device 40 for manufacturing the cell-derived vesicles according to the fourth embodiment of the present invention, and Table 1 showed results of crude-CDV mass production according to Experimental Examples 20 to 22.

TABLE 1

| | | Average | | | | | | |
|---|---|---|---|---|---|---|---|---|
| | | time of | | | | | | |
| | | once | | Particle | | | | Produc- |
| | | extrusion | Recovery | concentration | | | DNA | tion |
| | dP | process | weight | (×10¹¹ | Size | | (μg/ | yield |
| Division | (bar) | (sec) | (kg) | particles/mL) | (nm) | PDI | mL) | Y_{P/C} |
| Exp. Ex. 20 | 0.20 | 36.8 | 17.3 | 0.84 | 159.1 | 0.285 | 4.80 | 17,920 |
| Exp. Ex. 21 | 0.26 | 28.3 | 18.4 | 0.63 | 159.3 | 0.314 | 5.02 | 13,440 |
| Exp. Ex. 22 | 0.19 | 30.9 | 18.7 | 0.69 | 164.6 | 0.333 | 4.80 | 14,720 |
| Mean | 0.22 | 32.0 | 18.1 | 0.72 | 161 | 0.311 | 4.87 | 15,360 |
| STDEV | 0.04 | 4.4 | 0.7 | 0.11 | 3 | 0.024 | 0.13 | 2,308 |
| % CV | 17.5 | 13.6 | 4.1 | 15.0 | 1.9 | 7.8 | 2.6 | 15.0 |

dP = Average of maximum and minimum pressure differences measured in extrusion solution, during extrusion

### Experimental Example 19

For cell extrusion, a total of four cartridge depth filters (Sartopure PP3 Cartridge, total membrane area: 1.8 m², retention rates: 0.65 μm) were mounted in a filter assembly. Specifically, two cartridge depth filters were mounted on each lower part included in the two filter assemblies, and an idle mounting holes was closed with a cap. Thereafter, a gas supply line for supplying nitrogen (N₂) gas was connected to each filter assembly. 12.5 L of a HEK293 cell culture solution at a concentration of 6.00E+06 cells/mL was added to the two filter assemblies in half. The spent media were discharged to a harvest line by opening both valves of the gas supply line and pressurizing at 0.4 bar. The discharge time of the spent media was 5 minutes. As a result of analyzing the spent media using the method for analyzing whether cells remained in the spent media described in Experimental Example 5, it was analyzed that there were no cells remaining in the spent media. 20 L of 37° C. PBS was back-flushed into the harvest line using a peristaltic pump. The back-flushing time was total 16 minutes. For extrusion, the resuspended cell solution passed through the cartridge filter inside the filter assembly in one direction by closing one valve of the gas supply line and opening the other valve thereof. The resuspended cell solution passed through the cartridge filter total 70 times by repeating the opening and closing of the left and right valves in reverse. The nitrogen gas pressure supplied to the filter assembly was 1.0 bar for the first extrusion and 2.5 bar for extrusion 2 to 70 times. The average time required for one extrusion was measured as 32.2 seconds. The particle concentration, particle size, PDI, and DNA concentration in the final extrusion solution were measured, and the production yield of particles per cell was analyzed as 17,493 particles/cell.

### Experimental Example 20

For cell extrusion, a total of four cartridge depth filters (Sartopure PP3 Cartridge, total membrane area: 1.8 m², retention rates: 0.65 μm) were mounted in a filter assembly. Specifically, two cartridge depth filters were mounted on each lower part included in the two filter assemblies, and an idle mounting hole was closed with a cap. Thereafter, a gas supply line for supplying nitrogen (N₂) gas was connected to each filter assembly. 12.5 L of a HEK293 cell culture solution at a concentration of 6.00E+06 cells/mL was added to the two filter assemblies in half. The spent media were discharged to a harvest line by opening both valves of the gas supply line and pressurizing at 0.4 bar. The discharge time of the spent media was 22 minutes. As a result of analyzing the spent media using the method for analyzing whether cells remained in the spent media described in Experimental Example 5, it was analyzed that there were no cells remaining in the spent media. 16 L of 37° ° C. PBS was back-flushed into the harvest line using a peristaltic pump. The back-flushing time was total 9 minutes. For extrusion, the resuspended cell solution passed through the cartridge filter inside the filter assembly in one direction by closing one valve of the gas supply line and opening the other valve thereof. The resuspended cell solution passed through the cartridge filter total 70 times by repeating the opening and closing of the left and right valves in reverse. The nitrogen gas pressure supplied to the filter assembly was 1.0 bar for the first extrusion and 2.5 bar for extrusion 2 to 70 times. The average time required for one extrusion was measured as 36.8 seconds. The particle concentration and the particle size in the final extrusion solution were measured, and the production yield of particles per cell was analyzed as 17,920 particles/cell.

### Experimental Example 21

For cell extrusion, a total of four cartridge depth filters (Sartopure PP3 Cartridge, total membrane area: 1.8 m², retention rates: 0.65 μm) were mounted in a filter assembly. Specifically, two cartridge depth filters were mounted on each lower part included in the two filter assemblies, and an idle mounting hole was closed with a cap. Thereafter, a gas supply line for supplying nitrogen (N₂) gas was connected to each filter assembly. 12.5 L of a HEK293 cell culture solution at a concentration of 6.00E+06 cells/mL was added to the two filter assemblies in half. The spent media were discharged to a harvest line by opening both valves of the gas supply line and pressurizing at 0.4 bar. The discharge time of the spent media was 19 minutes. As a result of analyzing the spent media using the method for analyzing whether cells remained in the spent media described in Experimental Example 5, it was analyzed that there were no cells remaining in the spent media. 16 L of 37° ° C. PBS was back-flushed into the harvest line using a peristaltic pump. The back-flushing time was total 9 minutes. For extrusion, the resuspended cell solution passed through the cartridge filter inside the filter assembly in one direction by closing one valve of the gas supply line and opening the other valve thereof. The resuspended cell solution passed through the cartridge filter total 70 times by repeating the opening and closing of the left and right valves in reverse. The nitrogen gas pressure supplied to the filter assembly was 1.0 bar for the first extrusion and 2.5 bar for extrusion 2 to 70 times. The average time required for one extrusion was measured as 28.3 seconds. The particle concentration, particle size, PDI, and DNA concentration in the final extrusion solution were measured, and the production yield of particles per cell was analyzed as 13,440 particles/cell.

### Experimental Example 22

For cell extrusion, a total of four cartridge depth filters (Sartopure PP3 Cartridge, total membrane area: 1.8 m², retention rates: 0.65 μm) were mounted in a filter assembly. Specifically, two cartridge depth filters were mounted on each lower part included in the two filter assemblies, and an idle mounting hole was closed with a cap. Thereafter, a gas supply line for supplying nitrogen (N₂) gas was connected to each filter assembly. 12.5 L of a HEK293 cell culture solution at a concentration of 6.00E+06 cells/mL was added to the two filter assemblies in half. The spent media were discharged to a harvest line by opening both valves of the gas supply line and pressurizing at 0.4 bar. The discharge time of the spent media was 21 minutes. As a result of analyzing the spent media using the method for analyzing whether cells remained in the spent media described in Experimental Example 5, it was analyzed that there were no cells remaining in the spent media. 16 L of 37° ° C. PBS was back-flushed into the harvest line using a peristaltic pump. The back-flushing time was total 9 minutes. For extrusion, the resuspended cell solution passed through the cartridge filter inside the filter assembly in one direction by closing one valve of the gas supply line and opening the other valve thereof. The resuspended cell solution passed through the cartridge filter total 70 times by repeating the opening and closing of the left and right valves in reverse. The nitrogen gas pressure supplied to the filter assembly was 1.0 bar for the first extrusion and 2.5 bar for extrusion 2 to 70 times. The average time required for one extrusion was measured as 30.9 seconds. The particle concentration, particle size, PDI, and DNA concentration in the final extrusion solution were measured, and the production yield of particles per cell was analyzed as 14,720 particles/cell.

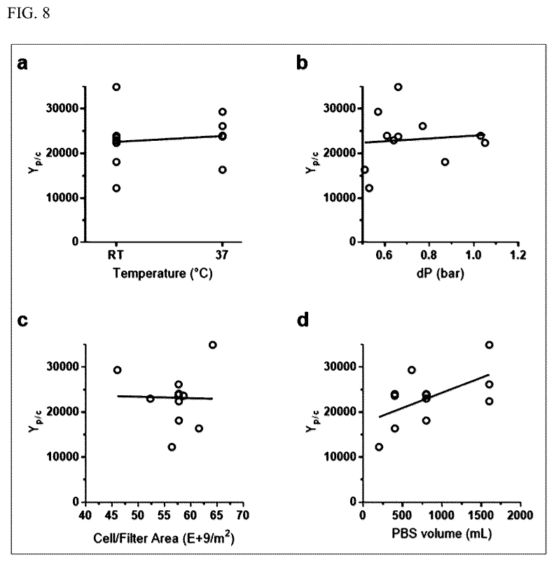

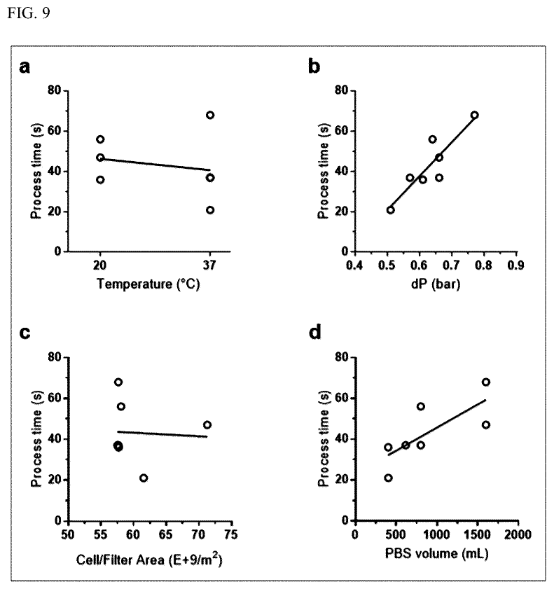

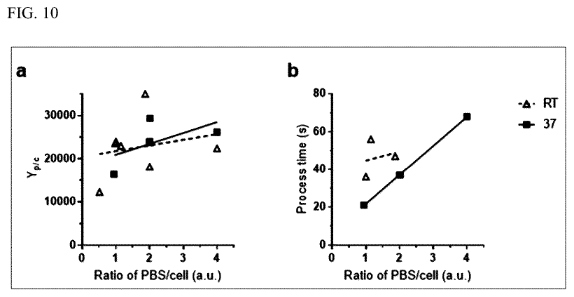

Meanwhile, FIG. 8 is a diagram of results for the production yield of particles per cell according to various parameters, FIG. 9 is a diagram of results for average time of once extruding process according to various parameters, and FIG. 10 is a diagram of results for production yield and average time of once extruding process according to a PBS amount per cell and a PBS temperature. As shown in the drawings, it may be confirmed that mass production of cell-derived vesicles is enabled with high efficiency using the device for manufacturing the cell-derived vesicles of the present invention.

### Experimental Example 23. Effect of Manufacturing Uniform Cell-Derived Vesicles

An experiment was conducted to determine whether an extrusion process using a depth filter was not only scaled up, but also uniform cell-derived vesicles were manufactured. An experimental group in which sequential extrusion was performed using a membrane as an extrusion filter was set as a comparative group.

For cell extrusion, two depth filters (Sartopure PP3 Capsules, total membrane area: 0.052 m², retention rates: 0.65 μm) were connected between pressure vessels, and a gas supply line for supplying nitrogen (N₂) gas was connected to each pressure vessel. The resuspended cell solution passed through the filter total 70 times in reverse.

The extrusion process using membrane filters made of polycarbonate was performed sequentially using membrane filters with pore sizes of 10 μm, 3 μm, and 0.4 μm, respectively.

HEK293 cell culture (6.17E+06 cells/mL) was used equally for the depth filter extrusion method and the membrane sequential extrusion method. After the extrusion process, the cells were treated with Benzonase enzyme at 37° C. for 1.5 hours to degrade the DNA from the extrusion. This was followed by Tangential Flow Filtration and Size Exclusion Chromatography. Finally, Amicon (3 kDa) was treated to concentrate and the final product was prepared using a 0.2 μm sterilization filter. After the extrusion process, the separation and purification process was the same for both the depth filter and membrane filter methods.

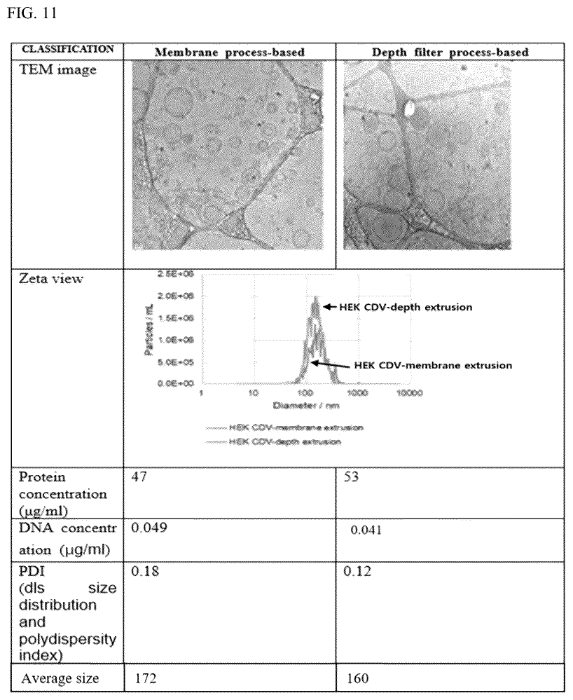

For CDVs produced according to the membrane sequential extrusion method or the depth filter extrusion method of the present invention, results of comparing the appearance, shape distribution, size distribution, and final protein and total DNA contents were illustrated in FIG. 11.

As illustrated in FIG. 11, it was confirmed that the appearance, shape distribution, size distribution, and final protein and total DNA contents were similar, but when using the depth filter, PDI was 0.12, which was lower than 0.18, PDI of the comparative group using the membrane filter process, and thus CDVs may be manufactured in a more monodispersed form.

As described above, the device for manufacturing cell-derived vesicles of the present invention and the manufacturing method using the same are very beneficial in that the cell-derived vesicles are manufactured in an environment with minimal external exposure to reduce the risk of contamination and deformation, to be manufactured stably, economically, and in large quantities, and to easily adjust the production capacity if necessary.

The above description just illustrates the technical spirit of the present invention and various changes, modifications, and substitutions may be made by those skilled in the art to which the present invention pertains without departing from an essential characteristic of the present invention. Therefore, the embodiments and the accompanying drawings disclosed in the present invention are used to not limit but describe the technical spirit of the present invention and the scope of the technical spirit of the present invention is not limited by the embodiments and the accompanying drawings. The protective scope of the present invention should be construed based on the following claims, and all the technical concepts in the equivalent scope thereof should be construed as falling within the scope of the present invention.

1. A method for manufacturing cell-derived vesicles using a depth filter, comprising:
a) extruding cells through a depth filter; and
b) obtaining cell-derived vesicles from the extruded solution.

2. The method for manufacturing the cell-derived vesicles of claim 1, further comprising:
before the extruding step of step a),
a-1) harvesting the cells by passing a cell culture solution containing the cells through the depth filter; and
a-2) resuspending the harvested cells by supplying a cell suspension,
wherein the cells in step a) are cells in the cell suspension.

3. The method for manufacturing the cell-derived vesicles of claim 2, wherein the harvesting step, the resuspending step, and the extruding step are performed in one device.

4. The method for manufacturing the cell-derived vesicles of claim 2, wherein than the extruding step of step a) is performed at a higher pressure than the harvesting step of step a-1).

5. The method for manufacturing the cell-derived vesicles of claim 2, wherein the harvesting step of step a-1) is performed under pressure conditions of is 0.1 to 0.4 bar.

6. The method for manufacturing the cell-derived vesicles of claim 1, wherein the extruding step of step a) is performed under pressure conditions of 1 to 2.5 bar.

7. The method for manufacturing the cell-derived vesicles of claim 2, wherein the cell suspension is supplied in a reverse direction of the direction in which the cell culture solution is discharged from the depth filter.

8. The method for manufacturing the cell-derived vesicles of claim 1, wherein the extruding step of step a) is repeated 1 to 120 times.

9. The method for manufacturing the cell-derived vesicles of claim 8, wherein the repetition is performed by reciprocating through one depth filter.

10. The method for manufacturing the cell-derived vesicles of claim 8, wherein the repetition is performed by changing the pressure.

11. The method for manufacturing the cell-derived vesicles of claim 10, wherein the manufacturing method is performed by changing the pressure to 1 bar as a first pressure and 2.5 bar as a second pressure.

12. The method for manufacturing the cell-derived vesicles of claim 3, wherein the device comprises:
a first pressure vessel and a second pressure vessel, of which a culture solution supply line to which a cell culture solution is supplied and a gas supply line are connected to one side;
a first extrusion line connected to the other side of the first pressure vessel;
a second extrusion line which is connected to the other side of the second pressure vessel and communicates with the first extrusion line;
at least one depth filter which is disposed on the first extrusion line or the second extrusion line and through which the cell culture solution passes; and
a harvesting line which communicates with the first extrusion line and the second extrusion line.

13. The method for manufacturing the cell-derived vesicles of claim 12, wherein at least one depth filter is disposed on the first extrusion line and the second extrusion line, respectively.

14. The method for manufacturing the cell-derived vesicles of claim 13, wherein the device for manufacturing the cell-derived vesicles comprises:
a first filter assembly and a second filter assembly, on one side of which a culture solution supply line to which a cell culture solution is supplied and a gas supply line are connected, and in which a depth filter is provided;
a first extrusion line connected to the other side of the first filter assembly;
a second extrusion line which is connected to the other side of the second filter assembly and communicates with the first extrusion line; and
a harvesting line which communicates with the first extrusion line and the second extrusion line.

15. The method for manufacturing the cell-derived vesicles of claim 14, wherein the first filter assembly and the second filter assembly comprise:
a lower portion in which at least one mounting hole mounted with the depth filter is formed;
a middle portion which is formed so that the depth filter is contained therein and coupled to the lower portion; and
an upper portion which is fastened to the upper end of the middle portion and seals the inside together with the lower portion and the middle portion.

16. The method for manufacturing the cell-derived vesicles of claim 15, wherein the first filter assembly and the second filter assembly further comprise:
a cap which is provided to be detachable from the mounting hole and seals the mounting hole while inserted into the mounting hole.

17. A method for manufacturing cell-derived vesicles with reduced risk of exposure to external contamination comprising:
a) harvesting cells by passing a cell culture solution containing the cells through a depth filter;
b) resuspending the harvested cells by supplying a cell suspension;
c) extruding the cells resuspended in step b) through a depth filter; and
d) obtaining cell-derived vesicles from the extruded solution,
wherein steps a) to d) are performed in one device.

18. The method for manufacturing the cell-derived vesicles of claim 17, wherein the manufacturing method is performed in one device to reduce the risk of cells or cell-derived vesicles to be exposed to an external environment to be contaminated or deformed.

19. The method for manufacturing the cell-derived vesicles of claim 17, wherein the manufacturing method is performed in one device to improve a process rate.

20. The method for manufacturing the cell-derived vesicles of claim 17, wherein the device comprises:
a first pressure vessel and a second pressure vessel, of which a culture solution supply line to which a cell culture solution is supplied and a gas supply line are connected to one side;
a first extrusion line connected to the other side of the first pressure vessel;
a second extrusion line which is connected to the other side of the second pressure vessel and communicates with the first extrusion line;
at least one depth filter which is disposed on the first extrusion line or the second extrusion line and through which the cell culture solution passes; and
a harvesting line which communicates with the first extrusion line and the second extrusion line.
